(19) United States
(12) Patent Application Publication
(10) Pub. No.: US 2009/0176968 A1
(43) Pub. Date: Jul. 9, 2009

(54) Preparation of Triazole Containing Metal Chelating Agents

(75) Inventors: Thomas Mindt, Basel (CH); Roger Schibli, Baden (CH)

Correspondence Address: LERNER GREENBERG STEMER LLP, P O BOX 2480, HOLLYWOOD, FL, 33022-2480, US

(73) Assignee: PAUL SCHERRER INSTITUT, Villigen PSI (CH)

(21) Appl. No.: 12/305,986

(22) PCT Filed: Jun. 1, 2007
(86) PCT No.: PCT/EP07/04849

§ 371 Date: Jan. 27, 2009

(30) Foreign Application Priority Data
Jun. 20, 2006 (EP) 06 012 597.8

(51) Int. Cl. C07F 13/00; C07F 15/00; C07K 2/00; C07H 19/056; C07H 21/02; C07H 21/04; C08B 37/00; C09K 3/00

(52) U.S. Cl. 530/345; 534/14; 548/101; 530/400; 536/23.1; 536/22.1; 536/55; 252/625

(57) ABSTRACT

New chelating agents as well as their tricarbonyl complexes with technetium and rhenium and the use of these compounds in radiodiagnosis and radiotherapy are described. As a peculiarity of this invention, synthesis and coupling of the chelating systems to (bio)molecules is performed simultaneously. The new chelating agents are coupled to substances that accumulate in the diseased/targeted tissue.

The invention relates to the field of radiopharmaceuticals and describes new chelating agents as well as their tricarbonyl complexes with technetium and rhenium.

In recent years, the demand for radiodiagnostic agents that accumulate specifically in diseased tissues has significantly increased. This can be achieved if the radionuclide can be coupled to substances that accumulate selectively in sites of interest. Such radiotracers make molecular processes visible and trackable over time in a non-invasive manner in live animals and humans. Therefore, the stable and efficient incorporation of readily available radionuclides with optimal decay characteristics into molecules of diagnostic and therapeutic interest is of outmost importance for the development of novel radiotracers. The isotope technetium-99m is still the mainstay of routine diagnostic nuclear medicine. ^{99m}Tc is especially well suited for in-vivo use because of its advantageous physical properties (no corpuscular radiation, short half-life of 6.02 h, good detectability by its 140 KeV γ-radiation) and its broad availability. Technetium's higher homologue rhenium possesses two radionuclides (Re-186: T_{1/2}=90 h, βₘₐₓ⁻=1.1 MeV; Re-188: T_{1/2}=17 h, βₘₐₓ⁻=2.1 MeV) which are well suited for tumor therapy because of their corpuscular radiation.

To stably incorporate metallic radionuclides into molecules of interest It is necessary to provide bifunctional complexing agents that carry both functional groups for stable binding of the desired metal ion and one or more other functional groups for coupling the selectively accumulating molecule. Considerable efforts have been made to develop novel metal core for the radiolabeling of biomolecules with Tc-99m and Re-186/188 among which the organometallic precursor [M(OH₂)₃(CO)₃]⁺ (M=Tc, Re) is very prominent. The substitution behavior together with the electronic structure and the geometry of the precursor [M(OH₂)₃(CO)₃]⁺ (M=Tc, Re) is mainly responsible, that it forms very efficiently well defined and highly inert complexes with a wide variety of metal chelating systems. As a consequence a large variety of suitable bifunctional chelators for the functionalization of various biomolecules tailor-made for this novel M(CO)₃⁺-core have been designed. Most efficient ligand systems are based on amino acid scaffolds including cysteine, lysine and histidine. Particularly, ε-amino derivatized histidine, have proven to be excellent ligands for [M(OH₂)₃(CO)₃]⁺ (Pak et al. 2003). However, the preparation of amino acid-derived chelators still requires multi-step syntheses and the coupling strategies to biomolecules are limited with regards to cross-reactivity with other functional groups in the biomolecules. To overcome the shortcomings of previous methodologies, the synthetic complexity of the preparation of chelates and particularly (their attachment to) the functionalization of molecules of biomedical interest has to be significantly abridge.

The copper-(I)-catalyzed [3+2]cycloadditions of organic azides and terminal acetylenes yields a stable, 1,4-disubstituted 1,2,3-triazole linkage. This transformation, termed “click” reaction, has found tremendous resonance in different fields of chemical and biochemical research as well as in material sciences. Azides and alkynes easily installed by standard organic chemistry transformations as well as biochemically built into proteins. Since the reaction conditions are in general very mild they are suited for the modification of most biomolecules interesting for biomedical use. The 1,2,3-triazole moiety was design and is used till today for stable linkage of two (or more) chemical entity, without any further function.

Till today no systematic investigation of 1,4-disubstituted 1,2,3-triazoles as potential ligands for transition metals and in particular not for technetium and rhenium in the oxidation state +1. If the “click” reaction partners are chosen properly e.g. histidine like tridentate, or bis-triazole containing polyfunctional chelates useful for radiolabeling with [M(OH₂)₃(CO)₃]⁺. can be prepared under very mild conditions. One potential reason for the lack of 1,2,3-triazoles assisted coupling metal chelating entities to (bio)molecules via the “click” strategy is the fact that Cu(II) respectively Cu(I) (in catalytic amounts) is need for the efficient formation of 1,4-disubstituted 1,2,3-triazoles linkage. Cu(II)/Cu(I) react with a vide variety of polydentate ligands of the Werner type. As a consequence, the incorporation of such ligands of interest into biomolecules would be impaired in to ways:
1. Due to reactivity of the Cu(II)/Cu(I) with the Werner type of chelates the copper-ions will not be available in sufficient amount to accelerate the 1,2,3-triazoles formation. Excess Cu(I/II)-ions, extended reaction time and elevated temperature are presumably necessary achieve satisfying results. These conditions are not suitable for many biomolecules.
2. If copper-ions are used in excess to cope with the cross-reactivity mentioned above, most of the ligand will react with the copper-ions and form the corresponding, stable complexes. As a consequence the ligands will no longer be available for reaction with the metal ions/cores of interest, namely Tc/Re. Removal of the copper-ions is difficult and unpractical depending on the nature of the cheating system.

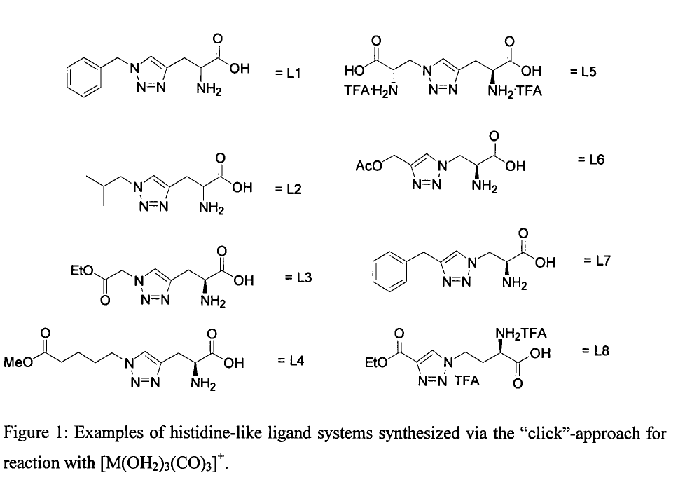

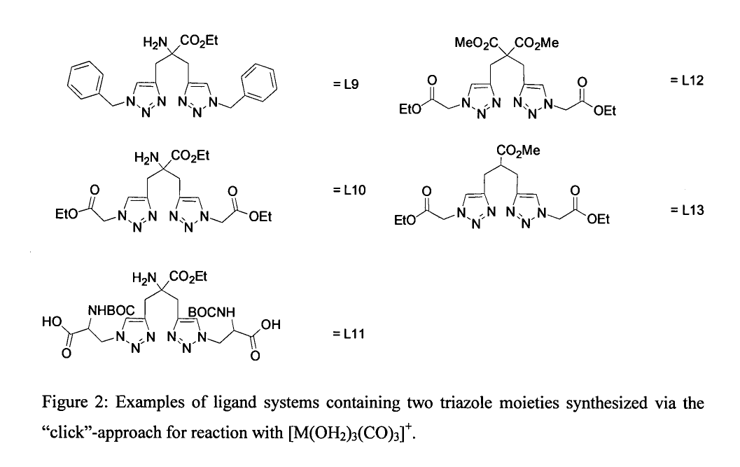

We have found that representatives of the chelating system described in claims 1-8 (FIGS. 1 and 2) reveal only a very low affinity for Cu(II)/Cu(I)-ions and can readily be removed from the reaction solution, e.g. with commercial metal scavengers. Cross-reactivity of the ligands L1-L13 with Cu(II)/Cu(I)-ions can be ignored or cause only minor problems. As a consequence the entire fraction of the synthesized ligand or the corresponding functionalized biomolecules is available for radiolabeling with the precursors [M(OH₂)₃(CO)₃]⁺. This is of outmost importance if one is using biomolecules, which are targeting specific receptors and high specific activities of the labeled biomolecule are necessary.

Preparation of the ligand systems L1-13 (FIGS. 1 and 2) were performed according to the procedure published by Sharpless et al. in very good yields. The reaction of the enantiomerically pure alkine (or azide) components with the azides (or alkine) gave rise to optically pure products. Reaction of the described ligand systems have been performed on the macroscopic as well as on the non-carrier-added level with Tc-99m and Re.

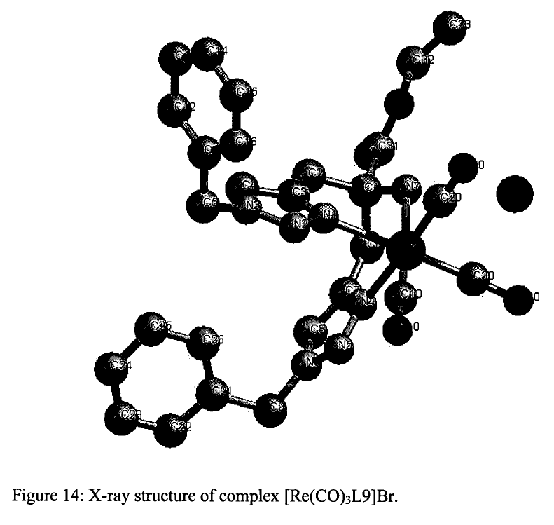

Reaction with rhenium gave rise to single species and well defined complexes with a 1:1 metal-to-chelate ration. Spectroscopic analyses provided evidence, that the chelates are coordinated in the tripodale fashion including on nitrogen of the 1,2,3-triazole moiety. This could be further proven by X-ray structure analysis of complex [Re(L9)(CO)₃] (FIG. 14).

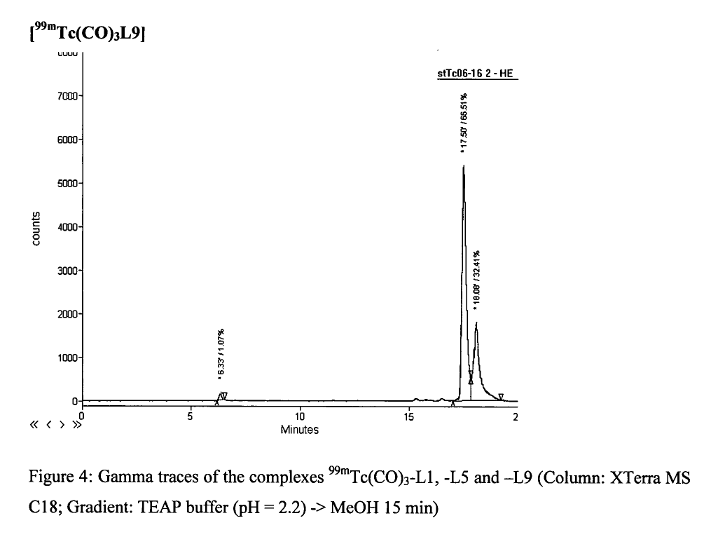

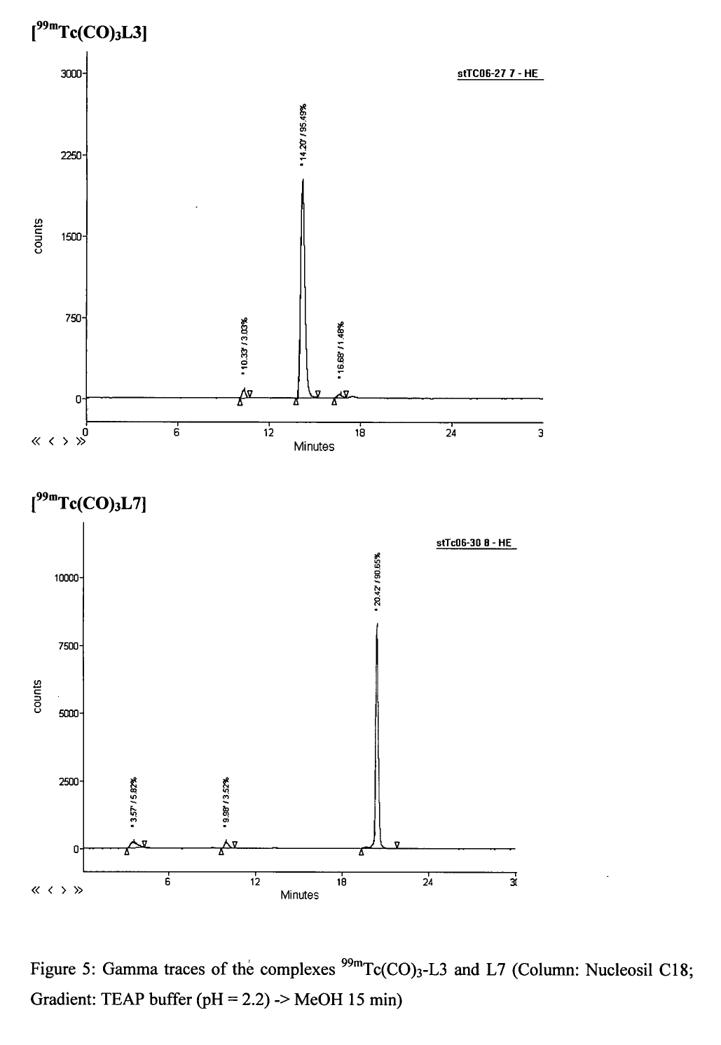

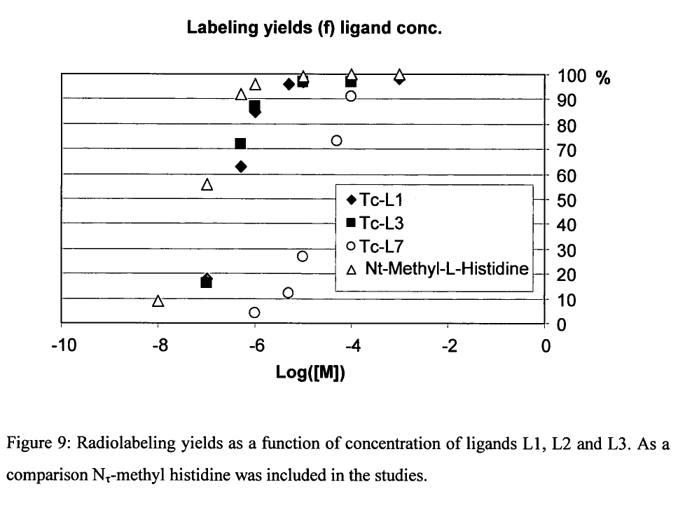

On the n.c.a. level the ligands were reacted with [^{99m}Tc(OH₂)₃(CO)₃]⁺ or [^{186/188} Re(OH₂)₃(CO)₃]⁺ in PBS buffer at pH=7.4 for 30 min or 60 min at 100° C. Ligand concentrations necessary to obtain the corresponding complexes with yields >90% varied between 5*10⁻6 M to 10⁻3 M (FIG. 9). It was generally observed, that concentration of the 1,2,3-triazole derivates with e.g. the methylglycine group at position C-4 (L1-L6) necessary to reach yields >90% were about one order of magnitude lower than those of the chelated with the methylglycine group at position 1 (L6-L8). The reactivity of e.g. L1-L3 were comparable with that of Nε-functionalized histidine derivatives e.g. Nε-methyl histidine (FIG. 9) this show the high potential of the triazole chelated presented in this invention. Radioactive traces of the Tc-99m complexes of selected ligands are shown in FIGS. 4 and 5.

In vitro stabilities of all the Tc-99m complexes in human plasma samples were >90% over a period of 24 h at 37° C.

Identity of the fully chemically and spectroscopically characterized rhenium complexes and the corresponding technetium species has been proven by comparison of the gamma-HPLC trace (Tc-99m) with the UV-HPLC trace (254 nm) of the rhenium complex.

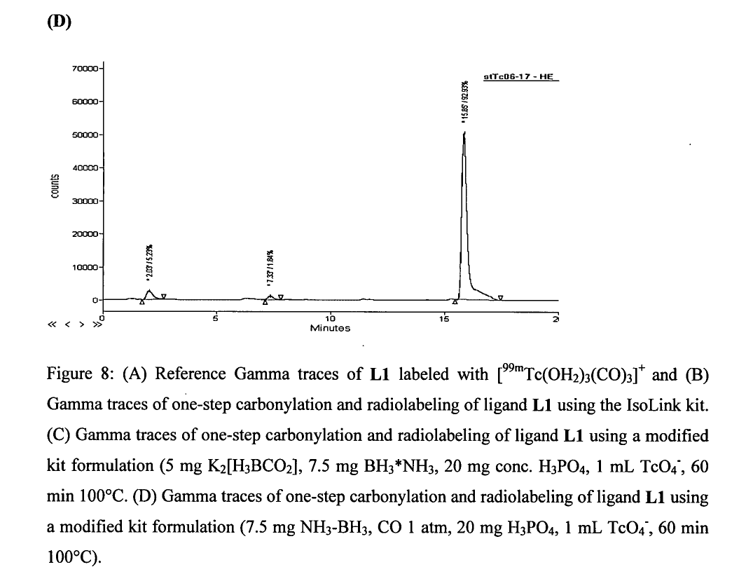

Alternatively the ^{99m}Tc(CO)₃-labeled products can be obtained in a single step starting from TcO₄⁻ using to kit-like preparations: (1) the IsoLink technology or (2) and (3) the alternative preparations described by Schibli et al. (Biojonjugate Chemistry, 2002) using K₂[H₃HCO₂] or CO_{(g)}, BH₃*NH₃ and H₃PO₄. Side products were observed in case of variant (1) whereas the HPLC trace of variant (2) and (3) gave identical results as the control experiment (FIG. 8).

However it is not only the merit of the present invention, disclosing an easy way of preparing novel tripodale ligand systems for the M(CO)₃⁺-core but the powerful perspective for future radiotracer development is the possibility to readily prepare a very powerful metal chelate while simultaneously incorporate it into any biomolecule as long as it comprise a azide or alkine functionality. A further intriguing feature of this invention is the fact, that for the synthesis of the chelates and their incorporation into a biologically active entity respectively no laborious protection/deprotection strategies are necessary in order to avoid cross-reactivity with other functional groups present in the biomolecule. Since click-reaction can be performed in aqueous as well as organic media virtually all types of biomolecules can be functionalized with one and the same strategy for later radiolabeling with the M(CO)₃⁺-core.

Considering further the synthetic simplicity of incorporation of an azide or an alkine group into an organic molecule of interests the functionalization strategy presented herein is of tremendous interest for the rapid development of new diagnostic tracers.

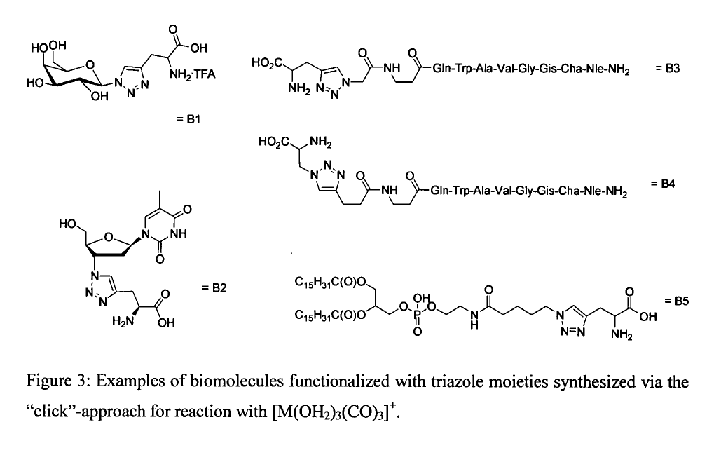

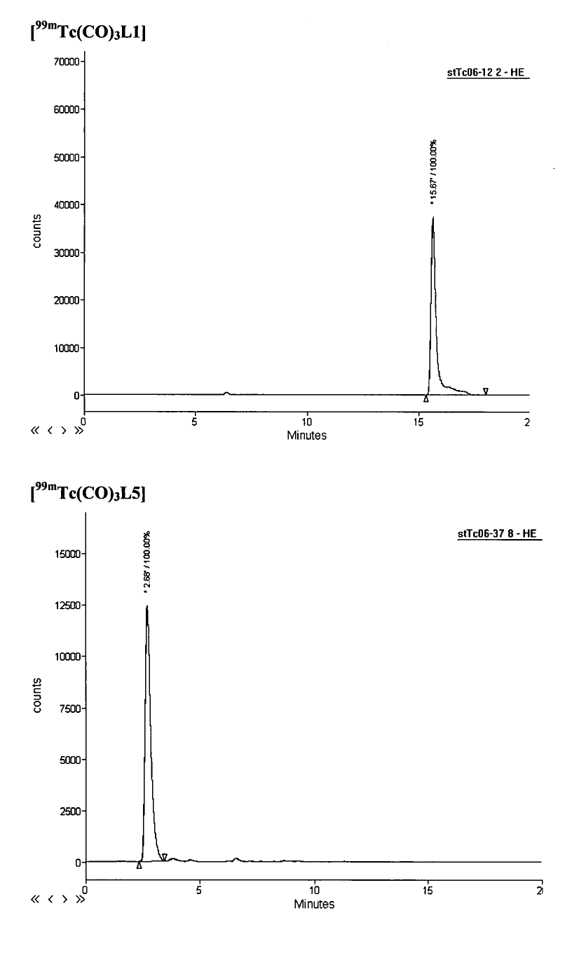

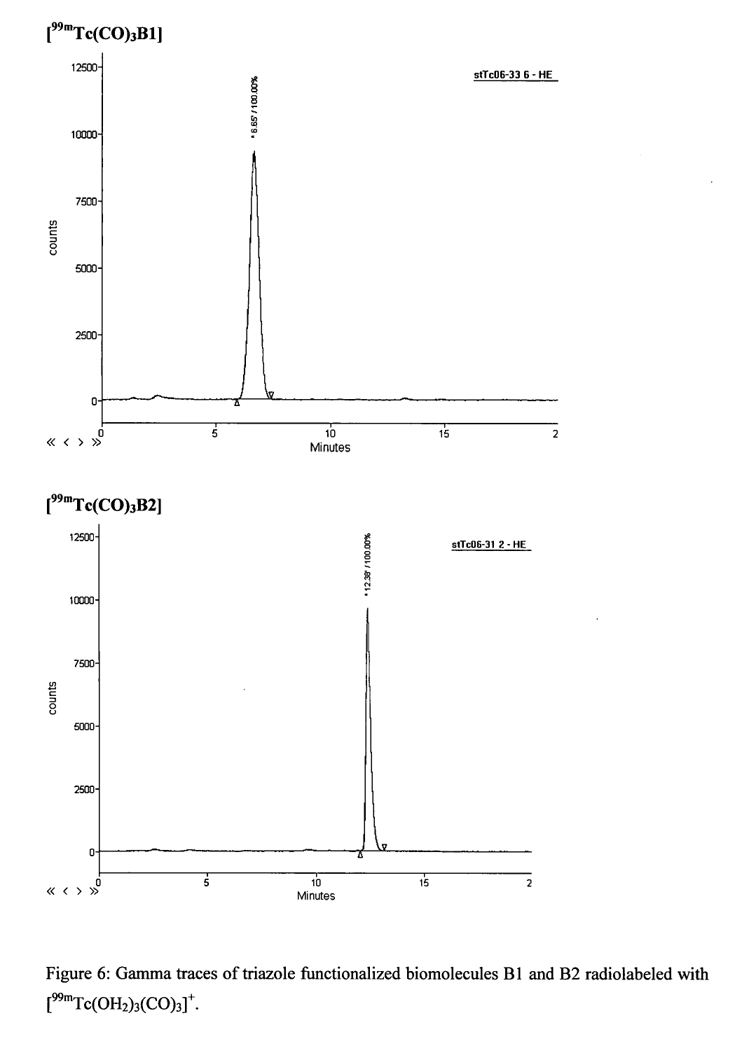

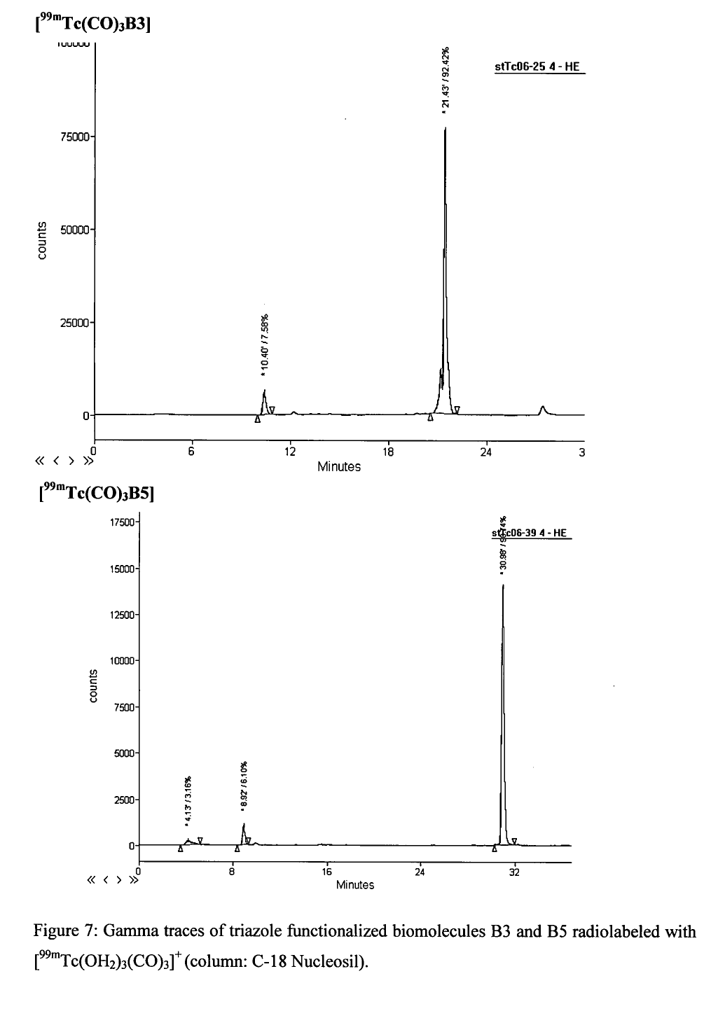

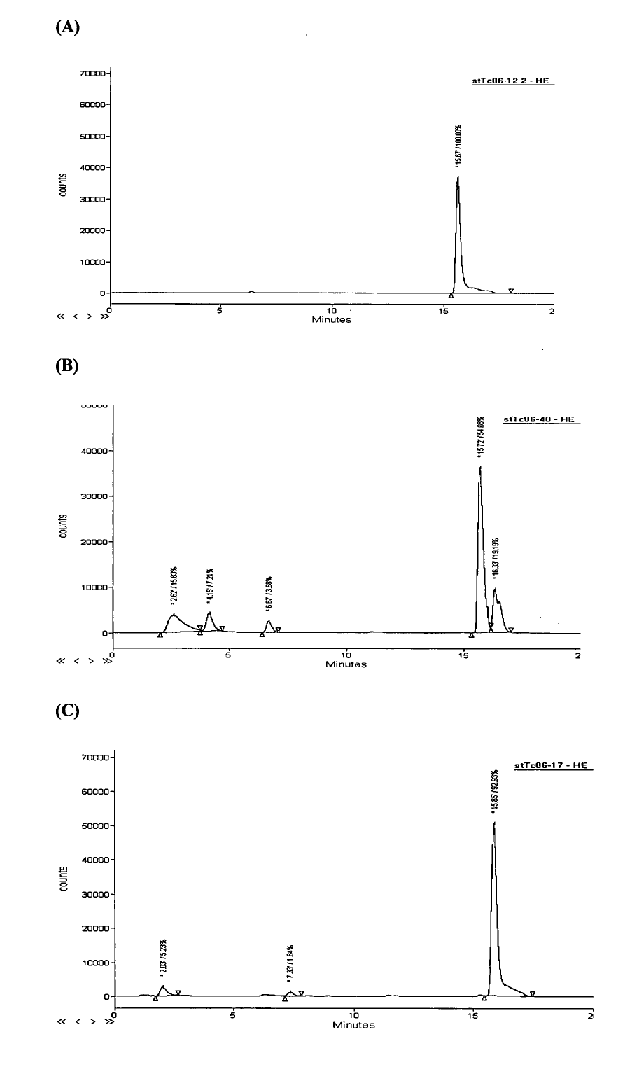

To prove these claims we selected four classes of (bio)molecules a carbohydrate derivative (1-Azido-1-deoxy-β-D-galactopyranose), a nucleoside derivative (3′-azido-thymidine), two peptide derivatives (azido-bombesin and akline Bombesin) and a phospholipid derivative (FIG. 3). In all of the examples the triazole containing, tridentate chelating system could be readily incorporated following the general synthetic “click” strategy. Reaction times varied between 2-12 hours in order to obtain yields were 40-90% (based in HPLC analyses). More intriguing, the fully functionalized and ready to label biomolecules were obtained in a single step. This was only possible because: (i) the orthogonal reactivity of the azide and alkine functionalities and (ii) none of the reactive components (biomolecule as well as the α-amino-carboxylate derivatives) and their functional groups needed to be protected or deprotected respectively. The versatility of the approach with respect to azide or alkine functionalized biomolecules could be exemplified in case of the Bombesin derivatives. The derivatives could be either reacted with azido-alanine or propargyl-glycine to give the corresponding 1,2,3-triazole chelate with the methylglycine group at position 1 or 4. Radiolabeling of the biomolecules B1-B5 was straightforward giving rise to single products in high yields (FIG. 6 and FIG. 7). Biological affinity was tested and proven in case of Bombesin derivatives B3 and B4. Affinity towards the cellular receptor was in vitro and in vivo fully retained.

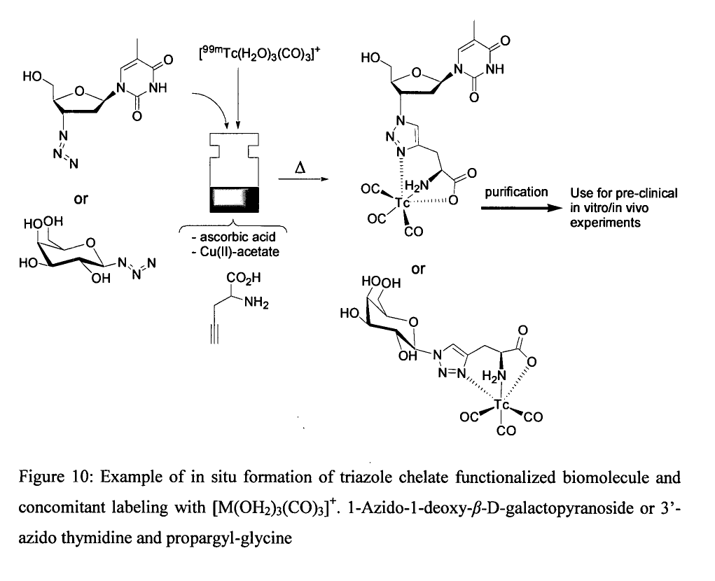

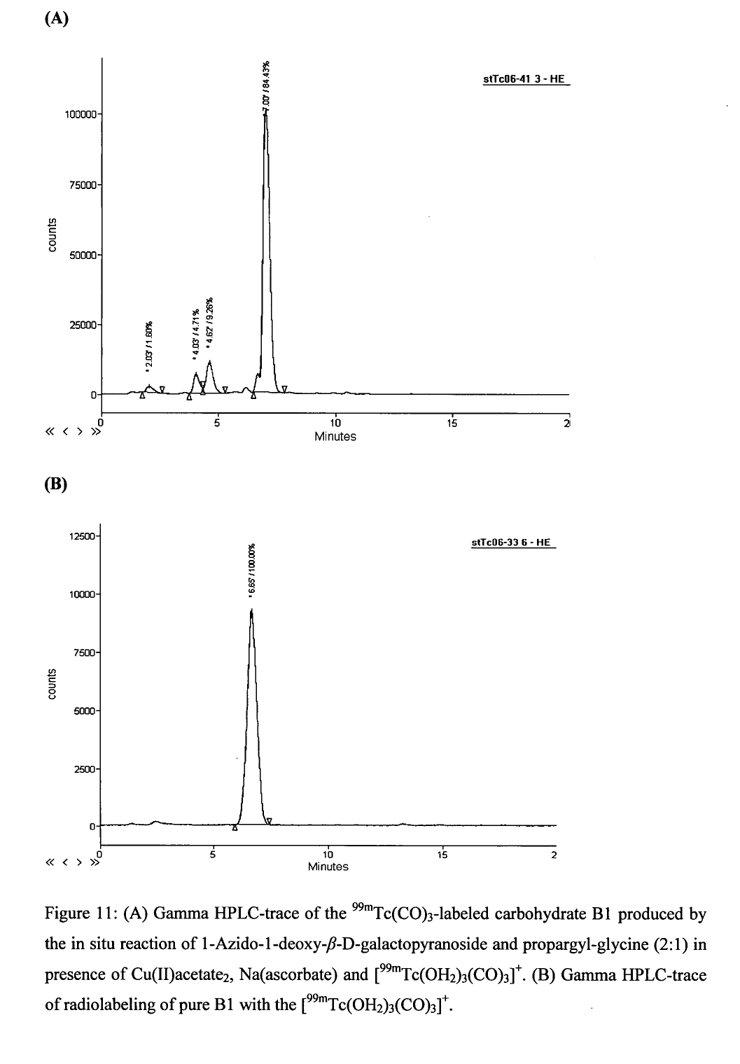

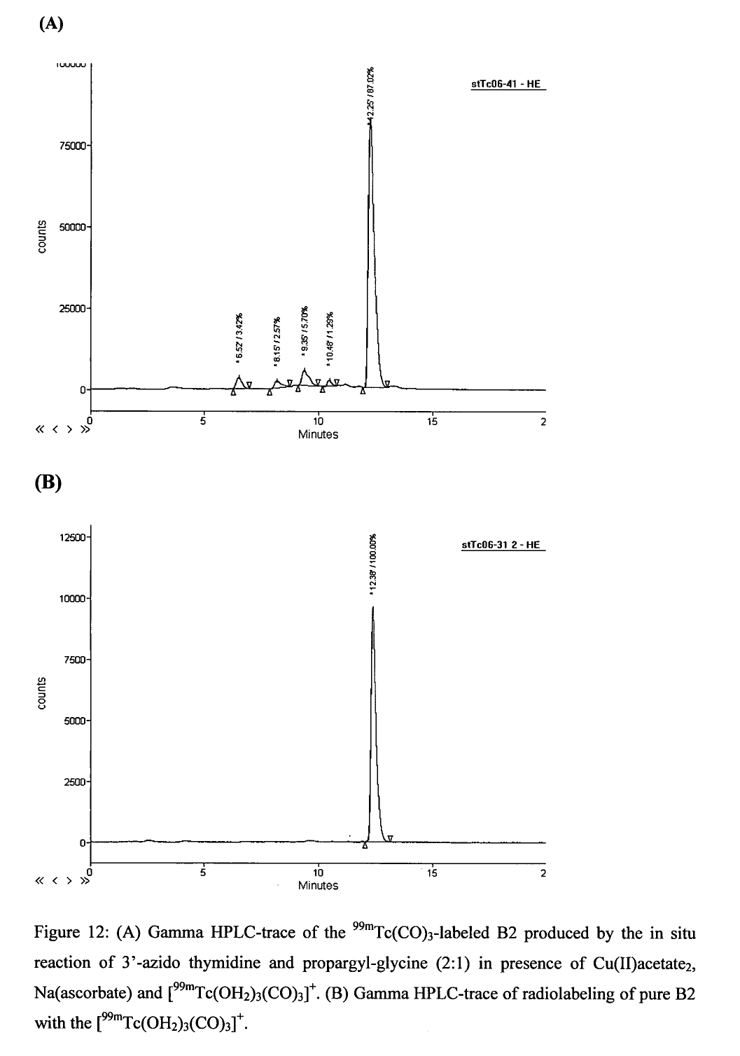

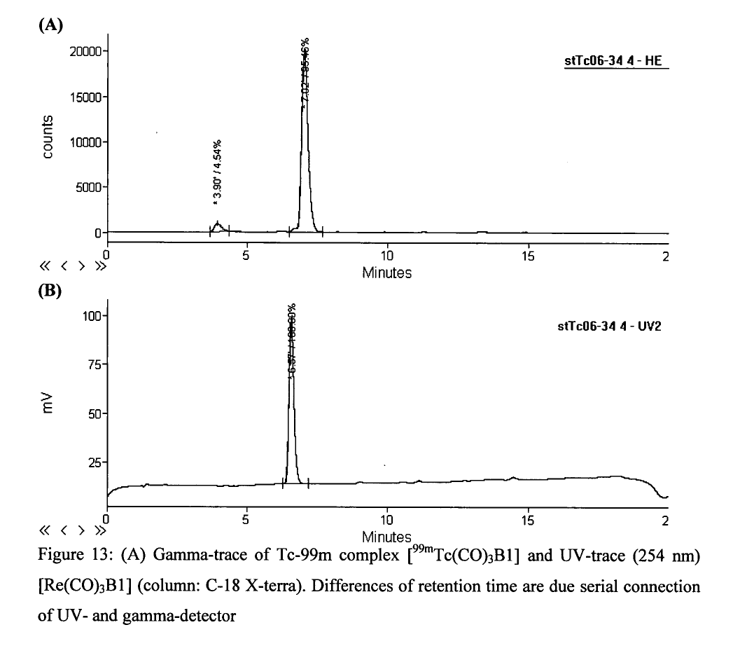

In case of 3′-azido thymidine and 1-Azido-1-deoxy-β-D-galactopyranose we could show that both compounds do not react with [^{99m}Tc(OH₂)₃(CO)₃]⁺. The same holds true for e.g. azide-alanine or propargyl glycine. On the other hand the crude reaction mixture of 3′-azido thymidine or 1-Azido-1-deoxy-β-D-galactopyranose with L-propargylglycine (in addition to Cu(II)-acetate and sodium ascorbate) incubated with [^{99m}Tc(OH₂)₃(CO)₃]⁺ resulted in products with identical retention time as those from the reaction with [^{99m}Tc(OH₂)₃(CO)₃]⁺ and pure 3′-[1,2,3-tirazole-4-methyl glycine]-thymidine or 1-[1,2,3-tirazole-4-methyl glycine]-1-deoxy-β-D-galactopyranose (FIG. 10). The orthogonal and selective reactivity of [^{99m}Tc(OH₂)₃(CO)₃]⁺ almost exclusively with the cyclisied triazole products but not with the individual starting materials is again a feature which is of interest (FIGS. 11 and 12). It enables and facilitates the preparation and radiolabeling of large series of potential tracer candidates without tedious pre-purification. Although the radiolabeling yields were slightly lower in case of the crude reaction solutions compared to the reaction starting from the pure ligands, this is not a critical issue since novel ^{99m}Tc-radiolabeled tracers are generally HPLC-purified before pre-clinical in vitro and in vivo testing. This way, whole libraries of different radiolabeled biomolecules comprising a triazole chelate can be readily prepared. Only the most promising candidates can be selected and prepared on a larger scale for further characterization. This approach minimizes consumption of eventually precious biological component as well as time to find “hits”. After purification on an analytical HPLC column these compounds can be used for further in vitro and in vivo testing.

### DISCLOSURE OF THE INVENTION

It is the merit of the present invention:
1. Disclosing an easy way of preparing ligand systems comprising one or more 1,2,3-triazole moieties useful for labeling with the M(CO)₃⁺-core.
2. Incorporation of the chelating systems into a (bio)molecule of interest for diagnostic or therapeutic purpose while synthesizing them simultaneously.
3. Functionalization of biomolecules with a metal chelating entities comprising one or more 1,2,3-triazole moieties without the necessity of any protection groups in aqueous or non-aqueous media.
4. Methods for the in situ preparation of 1,2,3-triazole functionalized (bio)molecules and subsequent radiolabeling with [M(H₂O)₃(CO)₃]⁺.
5. Method for the one-pot preparations of [M(H₂O)₃(CO)₃]⁺ radiolabeled 1,2,3-triazole functionalized (bio)molecules starting directly from [MO₄]⁻
6. The use of 1,2,3-triazole functionalized (bio)molecule radiolabeled with [M(H₂O)₃(CO)₃]⁺ for use as radio diagnostics or radiotherapeutics.

### EXAMPLES

### Syntheses of the Ligand Systems

### Example 1

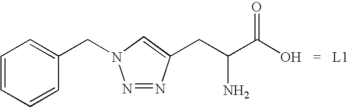

Compound L1. Benzylazide (53 mg, 0.4 mmol), N(α)-Boc-L-propargylglycine (85 mg, 0.4 mmol) copper(II)acetate (7 mg, 0.04 mmol) and sodium ascorbate (16 mg, 0.08 mmol) were mixed in t-butanol/water (1:1; 3.0 mL) and stirred at rt over night. The resulting green solution was diluted with ethylacetate (5 mL) and washed with brine (2×5 mL). The aqueous solutions were extracted with ethylacetate (2×5 mL). The organic extracts were combined, dried over Na₂SO₄ and concentrated under reduced pressure. The crude product was purified by flash chromatography on silica gel with mixtures of CH₂Cl₂/MeOH (4:1→2:1) to afford the intermediate as a pale yellow solid (86 mg, 62%): mp ° C.; IR (neat) v 3359, 2977, 2927, 1691, 1562, 1402, 1051 cm⁻¹; LRLC-MS: [M+H]+=347.05 (calc. for C₁₇H₂₂N₄O₄: 346.38); [α]_{D}²⁰=+11.0 (c=0.9 in CHCl₃). BOC-protected intermediate (65 mg, 0.19 mmol) was dissolved in CH₂Cl₂/TFA (2:1; 2.0 mL) and stirred at rt over night. Concentration under reduced pressure followed by repeated dissolving of the residue in MeOH and evaporation under reduced pressure provided the product as hygroscopic, white solid (68 mg, 98%): mp>195° C. (decomp.); IR (neat) v 3130, 2930, 2859, 1674, 1592, 1198, 1137, 718 cm⁻¹; ¹H-NMR (D₂O containing 0.5% DCl) δ 7.96 (s, 1H), 7.40-7.25 (m, 5H), 5.56 (s, 2H), 4.36 (t, 1H, J=6.2 Hz), 3.37 (d, 2H, J=6.2 Hz) ppm; ¹³C-NMR (D₂O containing 0.5% DCl) δ 170.4, 162.5 (q, J=36.0 Hz), 140.6, 134.4, 129.1, 128.8, 128.1, 125.4, 116.1 (q, J=291.8 Hz), 54.2, 52.2, 25.3 ppm; LRLC-MS: [M+H]+=247.06 (calc. for C₁₂H₁₄N₄O₂: 246.27).

### Example 2

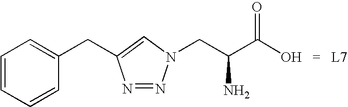

Compound L7: 3-Phenyl-1-propyne 93 μL, 87 mg, 0.75 mmol), N(α)-Boc-L-azidoalanine (173 mg, 0.75 mmol) copper(II)acetate (14 mg, 0.08 mmol) and sodium ascorbate (30 mg, 0.15 mmol) were mixed in t-butanol/water (1:1; 6.0 mL) and stirred at rt over night. The resulting green solution was diluted with ethylacetate (10 mL) and washed with brine (2×10 mL). The aqueous solutions were extracted with ethylacetate (2×5 mL). The organic extracts were combined, dried over Na₂SO₄ and concentrated under reduced pressure. The crude product was purified by flash chromatography on silica gel with mixtures of CH₂Cl₂/MeOH (4:1→2:1) to afford the product as a pale yellow solid (177 mg, 68%): mp>190° C. (decomp.); IR (neat) v 3206, 2980, 1688, 1602, 1368, 1190, 1151, 1066 cm⁻¹; ¹H-NMR (CD₃OD) δ 7.59 (s, 1H), 7.35-7.25 (m, 5H), 4.91-4.79 (m, partly covered by HDO signal, 1H, J=4.2 Hz), 4.62 (dd, 1H, J=13.6 and 7.1 Hz), 4.38 (dd, 1H, J=7.1 and 4.2 Hz), 4.00 (s, 2H), 1.33 (s, 9H) ppm; LRLC-MS: [M+H]+=347.02 (calc. for C₁₇H₂₂N₄O₄: 346.38); [α]_{D}³⁰=+24.7 (c=0.9 in CHCl₃). BOC-intermediate (113 mg, 0.33 mmol) was deprotected in CH₂Cl₂/TFA (2:1; 3.0 mL) give a hygroscopic, white solid (110 mg, 93%): mp>220° C. (decomp.); IR (neat) v 3363, 2977, 1710, 1674, 1198, 721 cm⁻¹; ¹H-NMR (D₂O containing 0.5% DCl) δ 8.05 (s, 1H), 7.29-7.13 (m, 5H), 5.03 (dd, 1H, J=15.3 and 5.4 Hz), 4.99 (dd, 1H, J=15.3 and 4.5 Hz), 4.64 (dd, 1H, J=5.4 and 4.5 Hz) 4.04 (s, 2H) ppm; ¹³C-NMR (D₂O containing 0.5% DCl) δ 167.9, 162.1 (q, J=37.0 Hz), 145.8, 129.0, 128.7, 127.3, 127.0, 115.8 (q, J=289.8 Hz), 51.9, 50.0, 29.5 ppm; LRLC-MS: [M+H]+=247.06 (calc. for C₁₂H₁₄N₄O₂: 246.27).

### Example 3

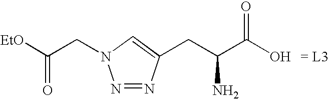

Compound L3. Azidoaceticacid ethylester (129 mg, 1.0 mmol), L-propargylglycine (113 mg, 1.0 mmol), copper(II)acetate (18 mg, 0.1 mmol) and sodium ascorbate (40 mg, 0.2 mmol) were mixed in t-butanol/water (1:1; 6.0 mL) and stirred at rt over night. QuadraPure-IDA® resin (0.2 g) was added and the mixture was gently shaken at rt for 2 h during which the blue color of the solution faded. The resulting brown solution was decanted and added drop wise to ethanol (100 mL). Filtration at 0° C. yielded compound L3 as a white powder (220 mg, 91%): mp 272-274° C.; IR (neat) v 3126, 2980, 2909, 1745, 1577, 1491, 14.09, 1220, 1197, 1061 cm⁻¹; ¹H-NMR (D₂O) δ 7.95 (s, 1H), 5.40 (s, 2H), 4.31 (q, 2H, J=7.2 Hz), 4.10 (t, 1H, J=6.4 Hz), 3.39 (dd, 1H, J=15.7 and 4.9 Hz), 3.36 (dd, 1H, J=15.7 and 7.1 Hz), 1.31 (t, 3H, J=7.2 Hz) ppm; ¹³C-NMR (D₂O) δ 173.1, 169.0, 142.1, 125.9, 63.4, 54.3, 51.0, 26.3, 13.2 ppm; LRLC-MS: [M+H]⁺=243.07 (calc. for C₉H₁₄N₄O₄: 242.23); elemental analysis (calculated %-values in parenthesis) C 44.51 (44.63), H 5.70 (5.83), N 22.88 (23.13), O 26.52 (26.42); [α]_{D}²⁰=−10.5 (c=1.0 in H₂O).

### Example 4

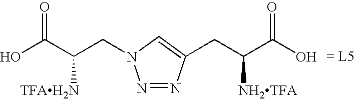

Compound L5 N(α)-Boc-L-azidoalanine (173 mg, 0.75 mmol), N(α)-Boc-L-propargylglycine (160 mg, 0.75 mmol) copper(II)acetate (14 mg, 0.08 mmol) and sodium ascorbate (30 mg, 0.15 mmol) were mixed in t-butanol/water (1:1; 6.0 mL) and stirred at rt over night. The resulting blue solution was concentrated under reduced pressure. The residue was purified by flash chromatography on silicagel with mixtures of CH₂Cl₂/MeOH (10:1→4:1) to afford the intermediate as a white solid (203 mg, 61%): mp>185° C. (decomp.); IR (neat) v 3385, 2976, 2905, 1684, 1603, 1397, 1055 cm⁻¹; ¹H-NMR (CD₃OD) δ 8.03 (bs, 1H), 4.87 (bs, 2H), 4.55-4.22 (m, 3H), 3.35-3.02 (m, 2H), 1.44 (s, 9H) ppm; LRLC-MS: [M+H]+=444.08 (calc. for C₁₈H₂₉N₅O₈: 443.45); Boc-intermediate (60 mg, 0.14 mmol) was deprotected in CH₂Cl₂/TFA (2:1; 3.0 mL) by the procedure described for compound L5 to give a hygroscopic, white solid (60 mg, 94%): mp>250° C. (decomp.); IR (neat) v 3302, 2973, 1738, 1705, 1663, 1516, 1212, 1162, 1051, 725 cm⁻¹; ¹H-NMR (D₂O containing 0.5% DCl) δ 7.91 (s, 1H), 4.98 (dd, 1H, J=15.4 and 5.4 Hz), 4.93 (dd, 1H, J=15.4 and 4.3 Hz) 4.60 (dd, 1H, J=5.4 and 4.3 Hz), 4.34 (t, 1H, J=6.1 Hz), 3.33 (d, 2H, J=6.1 Hz) ppm; ¹³C-NMR (D₂O containing 0.5% DCl) δ 170.6, 168.6, 162.6 (q, J=36.3 Hz), 141.3, 125.9, 116.1 (q, J=291.0 Hz), 52.5, 52.3, 48.7, 25.4 ppm; LRLC-MS: [M+H]+=244.05 (calc. for C₈H₁₃N₅O₄: 243.22); [α]20=−4.1 (c=1.0 in H₂O).

### Example 5

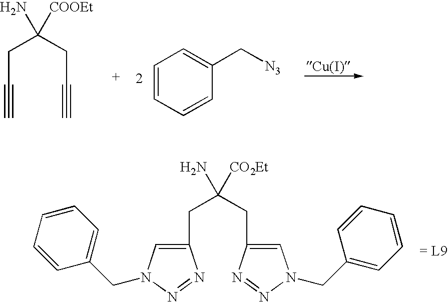

Compound L9: Dialkyne (90 mg, 0.5 mmol), benzyl azide (133 mg, 1.0 mmol), copper(II)acetate (182 mg, 1.0 mmol) and sodium ascorbate (396 mg, 2 mmo) were dissolved in tert-butanol/water (1:1, 8 mL) and the mixture was stirred at rt over night. The resulting green solution was diluted with ethyl acetate (20 mL) and washed with brine (15 mL), aqueous NH₄OH (5%; 2×15 mL) and again with brine (10 mL). The aqueous extracts were extracted with the ethyl acetate (2×20 mL). The organic extracts were combined, dried over Na₂SO₄ and concentrated under reduced pressure. The crude product was purified by chromatography on silicagel with CH₂Cl₂/MeOH (20:1) to yield compound L9 as a white powder (200 mg, 90%): mp 117-118° C.; IR (neat) 3132, 2983, 2933, 1727, 1454, 1215, 1045, 720 cm⁻¹; ¹H-NMR (CD₃OD) 7.74 (s, 2H), 7.40-7.25 (m, 10H), 5.57 (d, 2H, J=14.9 Hz), 5.53 (d, 2H, J=14.9 Hz), 3.91 (q, 2H, J=7.2 Hz), 3.23 (d, 2H, J=14.5 Hz), 2.97 (d, 2H, J=14.5 Hz), 0.99 (t, 3H, J=7.2 Hz) ppm; ¹³C-NMR (CD₃OD) 176.0, 144.3, 137.0, 130.2, 129.7, 129.2, 125.2, 62.7, 62.5, 55.0, 36.4, 14.4 ppm; LRMS: [M+H]+=446.11 (calc. for C₂₄H₂₇N₇O₂: 445.52).

### Example 6

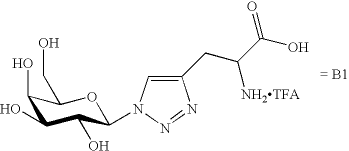

Compound B1. 1-Azido-1-deoxy-β-D-galactopyranoside tetraacetate (187 mg, 0.5 mmol), N(α)-Boc-L-propargylglycine (106 mg, 0.5 mmol) copper(II)acetate (9 mg, 0.05 mmol) and sodium ascorbate (20 mg, 0.10 mmol) were mixed in t-butanol/water (1:1; 4.0 mL) and stirred at rt over night. The resulting green solution was diluted with ethylacetate (10 mL) and washed with brine (2×10 mL). The aqueous solutions were extracted with ethylacetate (2×5 mL). The organic extracts were combined, dried over Na₂SO₄ and concentrated under reduced pressure. The crude product was purified by flash chromatography on silica gel with mixtures of CH₂Cl₂/MeOH (10:1→4:1) to afford the intermediate as a white solid (217 mg, 74%): mp>190° C. (decomp.); IR (neat) v 3406, 2977, 2934, 1752, 1684, 1588, 1395, 1366, 1215, 1254 cm⁻¹; ¹H-NMR (CD₃OD) δ 7.95 (s, 1H), 6.06 (d, 1H, J=9.2 Hz), 5.65 (t, 1H, J=9.8 Hz), 5.55 (d, 1H, J=2.7 Hz), 5.41 (dd, 1H, J=10.3 and 3.4 Hz), 4.46 (t, 1H, J=6.5 Hz), 4.30-4.15 (m, 2H), 4.12 (dd, 1H, J=11.4 and 6.9 Hz), 3.38-3.25 (m, partly covered by CD₃OD signal, 1H, J=5.0 Hz), 3.15 (dd, 1H, J=14.8 and 6.6 Hz), 2.21 (s, 3H), 2.02 (s, 3H), 2.00 (s, 3H), 1.86 (s, 3H), 1.42 (s, 9H) ppm; ¹³C-NMR (CD₃OD) δ 172.0, 171.9, 171.3, 170.6, 158.3, 146.5, 123.4, 87.0, 80.4, 75.0, 72.4, 69.6, 68.7, 62.6, 49.0, 29.6, 28.8, 20.6, 20.5, 20.4, 20.2 ppm (one quarternary carbon is not visible); LRLC-MS: [M+H]+=587.12 (calc. for C₂₄H₃₄N₄O₁₃: 586.55); Tetraacetate (152 mg, 0.26 mmol) was dissolved in methanol (2.0 mL) and a catalytic amount of sodium methoxide (1.4 mg, 0.03 mmol) was added. The solution was stirred at rt over night and then concentrated under reduced pressure to yield the product as a white solid (107 mg, 98%): mp>110° C. (decomp.); IR (neat) v 3345, 2980, 2930, 1681, 1592, 1398, 1162, 1090, 1054, 886 cm⁻¹; ¹H-NMR (CD₃OD) δ 7.99 (s, 1H), 5.54 (d, 1H, J=8.9 Hz), 4.27 (bs, 1H), 4.17 (t, 1H, J=9.1 Hz), 4.09 (bs, 1H), 3.90-3.18 (m, 4H), 3.29-3.07 (m, 2H), 1.29 (s, 9H) ppm; ¹³C-NMR (CD₃OD) δ 179.4, 157.8, 145.5, 123.4, 90.4, 80.7, 79.9, 75.3, 71.6, 70.5, 62.5, 56.6, 29.9, 28.9 ppm; LRLC-MS: [M+H]+=419.06 (calc. for C₁₆H₂₆N₄O₉: 418.40). BOC-intermediate (113 mg, 0.33 mmol) was deprotected in CH₂Cl₂/TFA (2:1; 3.0 mL) by the procedure described for compound B1 to give as an off-white solid: mp>145° C. (decomp.); IR (neat) v 3298, 2919, 1670, 1438, 1198, 1134, 1093, 1065, 725 cm⁻¹; ¹H-NMR (D₂O) δ 8.08 (s, 1H), 5.62 (d, 1H, J=9.2 Hz), 4.15 (t, 1H, J=9.6 Hz), 4.08-4.00 (m, 2H), 3.93 (t, 1H, J=5.9 Hz), 3.81 (dd, 1H, J=9.8 and 3.3 Hz), 3.71 (d, 2H, J=6.0 Hz), 3.34 (dd, 1H, J=15.7 and 5.2 Hz), 3.29 (dd, 1H, J=15.7 and 6.8 Hz) ppm; ¹³C-NMR (D₂O) δ 172.9, 163.0 (q, J=35.6 Hz), 142.2, 123.6, 116.3 (q, J=291.7 Hz), 87.9, 78.3, 72.9, 69.7, 68.5, 60.8, 54.2, 26.3 ppm; LRLC-MS: [M+H]+=319.03 (calc. for C₁₁H₁₈N₄O₇: 318.28); [α]_{D}²⁰=+5.5 (c=4.2 in MeOH).

### Example 7

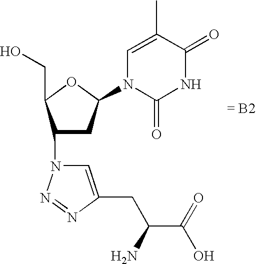

Compound B2: 3′-Azido-thymidine, N(α)-Boc-L-propargylglycine (106 mg, 0.5 mmol) copper(II)acetate (9 mg, 0.05 mmol) and sodium ascorbate (20 mg, 0.10 mmol) were mixed in t-butanol/water (1:1; 4.0 mL) and stirred at rt over night. The resulting green solution was diluted with ethylacetate (10 mL) and washed with brine (2×10 mL). The aqueous solutions were extracted with ethylacetate (2×5 mL). The organic extracts were combined, dried over Na₂SO₄ and concentrated under reduced pressure. The crude product was purified by flash chromatography on silica gel with mixtures of CH₂Cl₂/MeOH (10:1→4:1) to afford compound B2 as a white solid. LRLC-MS: [M+H]+=381.2 (calc. for C₁₅H₂₀N₆O₆: 380.14).

### Example 8

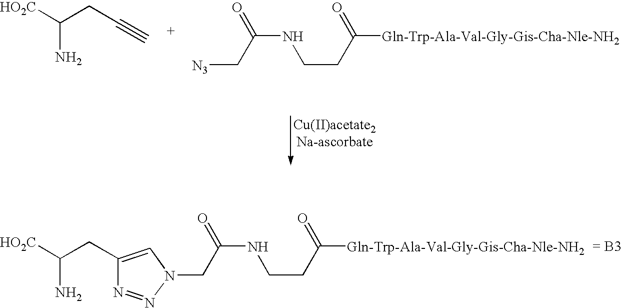

Compound B3: To a suspension of solid-phase supported Azido-peptide Gln-Trp-Ala-Val-Gly-Gis-Cha-Nle-NH₂ in DMF was added propargyl-glycine, copper(II)acetate (5 mg, 0.02 mmol) and sodium ascorbate (10 mg, 0.05 mmol). The reaction was stirred at room temperature for 12 h. Treatment of the bluish-green solution with QuadraPure-IDA® resin (0.5 g) for 4 days while gently shaking resulted in a pale yellow solution. The product was cleaved from the resin using a standard protocol. The raw product was purified via HPLC. MS: 1229.2 [M+1] (calc. for C₅₇H₈₄N₁₈O₁₃: 1228.4).

### Example 9

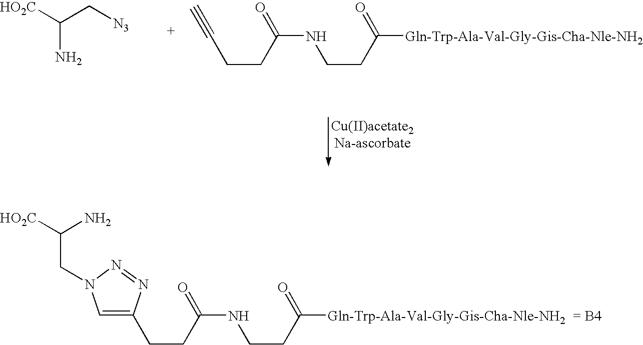

Compound B4: To a suspension of solid-phase supported alkine-peptide Gln-Trp-Ala-Val-Gly-Gis-Cha-Nle-NH₂ in DMF was added azido-alanine, copper(II)acetate (5 mg, 0.02 mmol) and sodium ascorbate (10 mg, 0.05 mmol). The reaction was stirred at room temperature for 12 h. Treatment of the bluish-green solution with QuadraPure-IDA® resin (0.5 g) for 4 days while gently shaking resulted in a pale yellow solution. The product was cleaved from the resin using a standard protocol. The raw product was purified via HPLC. MS: 1242.11 [M+] (calc. for C₅₈H₈₆N₁₈O₁₃: 1242.4).

### Example 9

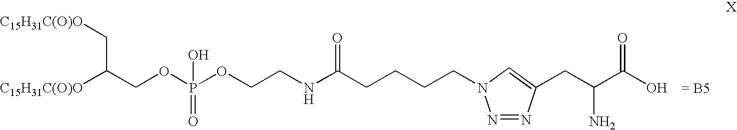

Compound B5: Azido-phospholipid (100 mg, 0.12 mmol), H-Pra-OH (14 mg, 0.12 mmol), copper(II)acetate (5 mg, 0.02 mmol) and sodium ascorbate (10 mg, 0.05 mmol) were mixed in t-butanol/water (1:1; 1.5 mL) and stirred at 50° C. for 8 hours. The resulting green solution was filtered and added to acetonitrile (100 mL). Filtration at 0° C. gave a green solid which was dissolved in hot THF (30 mL) and filtered through Celite. Treatment of the bluish-green solution with QuadraPure-IDA® resin (0.5 g) for 4 days while gently shaking resulted in a pale yellow solution. Filtration through Celite® and concentration under reduced pressure yielded compound B5 as a colorless oil (30 mg, 27%): LRLC-MS: [M+H]+=930.60 (calc. for C₄₂H₈₁N₄O₉P: 930.23);

### Example 10

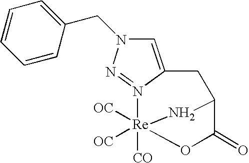

Ligand L1 (13 mg, 0.035 mmol) was dissolved in 5 mL 0.1 M HCl. [Re(CO)₃Br₃][NEt₄]₂ (30 mg, 0.038 mmol) was added and the mixture was stirred at 65° C. After 2 hours, the pH was increased to pH 5 with 1M NaOH. The reaction was followed by HPLC, and after stirring for 1 hour at pH 5 no further changes were observed. The solvent was removed under reduced pressure, and the residue redissolved in water. The product was purified with a Sep-Pak column (H₂O/methanol ratio 1:0, 4:1, 2:3, 1:1, 3:2, 4:1, 0:1). The fractions containing the product were combined and the solvent removed under reduced pressure to give the final product as a white powder (14 mg, 78%). ¹H NMR δ 3.25 (m, 2H), 4.06 (m, 1H), 5.20 (m, 1H), 5.64 (s, 2H), 5.88 (m, 1H), 7.39 (m, 5H), 7.94 (s, 1H). ¹³C NMR δ 27.47, 52.83, 56.01, 126.38, 129.62, 130.11, 130.21, 135.44, 144.08, 184.70, 196.74, 197.47, 198.27. IR (ATR diamond, cm⁻¹) 734, 1074, 1633, 1867, 1902, 2022, 2923. TOF-MS ES+: 516.9 (100%, [MH]⁺).

### Example 11

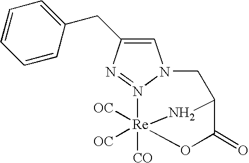

Ligand L7 (46 mg, 0.13 mmol) was dissolved in 15 mL 0.1 M HCl. [Re(CO)₃Br₃][NEt₄]₂ (100 mg, 0.13 mmol) was added and the mixture was stirred at 65° C. After 2 hours, the pH was increased to pH 5 with 1M NaOH. The reaction was followed by HPLC, and after stirring for 1 hour at pH 5 no further changes were observed. The solvent was removed under reduced pressure, and the residue redissolved in water. The product was purified with a Sep-Pak column (H₂O/methanol ratio 1:0, 4:1, 2:3, 1:1, 3:2, 4:1, 0:1). The fractions containing the product were combined and the solvent removed under reduced pressure to give the final product as a cream powder (37 mg, 55%). ¹H NMR δ 4.09 (s, 2H), 4.36 (m, 1H), 4.82 (m, 2H), 5.50 (m, 1H), 6.13 (m, 1H), 7.29 (m, 5H), 7.91 (s, 1H). ¹³C NMR δ 32.77, 53.70, 127.85, 128.82, 129.77, 129.80, 130.09, 139.30, 151.45, 181.70, 196.05, 197.05, 197.78. IR(ATR diamond, cm⁻¹) 729, 1147, 1643, 1876, 2025.

### Example 12

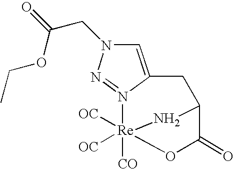

Ligand L3 (41 mg, 0.17 mmol) was dissolved in 30 mL ethanol. [Re(CO)₃Br₃][NEt₄]₂ (145 mg, 0.19 mmol) was added and the mixture was stirred at 50° C. The reaction was followed by HPLC, and after four hours no further changes were observed. The solvent was removed under reduced pressure, and the residue redissolved in water. The product was purified with a Sep-Pak column (H₂O/methanol ratio 1:0, 2:1, 1:1, 1:2, 0:1). The fractions containing the product were combined and the solvent removed under reduced pressure to give the final product as a white powder (45 mg, 52%). Found: C, 28.13; H, 2.77; N, 10.95. Calc. for C₁₂H₁₃N₄O₇Re: C, 28.18; H, 2.56; N, 10.95. ¹H NMR δ 1.30 (t, 3H), 4.09 (m, 1H), 4.27 (q, 2H), 5.41 (dd, 2H), 5.91 (m, 1H), 8.07 (s, 1H). ¹³C NMR δ 14.24, 14.46, 52.76, 63.50, 111.43, 128.30, 143.81, 167.43, 184.65, 196.61, 197.33, 198.04. IR (ATR diamond, cm⁻¹) 1220, 1376, 1636, 1747, 1871, 2025, 2337, 2360. TOF-MS ES+: 513.0 (100%, [MH]⁺).

### Example 13

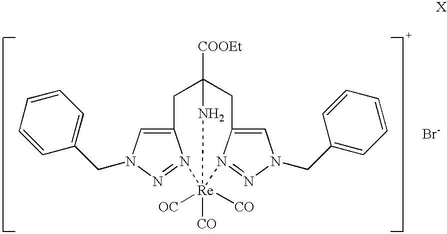

Double-click ligand L9 (45 mg, 0.1 mmol) and [Re(CO)₃(Br)₃][Et₄N]₂ (81 mg, 0.11 mmol) were suspended in dry ethanol (7 mL) and heated to 50° C. for 6 h. The resulting colourless solution was cooled to rt and concentrated under reduced pressure. The residue was purified by Sepak using first water and then water/methanol (3:1→1:1) to elute the product. Concentration of fractions containing the product (HPLC) under reduced pressure yielded, along with impure product (20 mg), the complex as a white powder (40 mg, 51%): mp 128-132° C.; IR (neat) 2028, 1903 (dominant signals of the carbonyl ligands), 1735, 1269, 729 cm⁻¹; ¹H-NMR (CD₃OD) 7.62 (s, 2H), 7.20-7.03 (m, 10H), 5.87 (bs, 1H), 5.29 (d, 2H, J=14.6 Hz), 5.23 (d, 2H, J=14.6 Hz), 4.11 (t, 2H, J=7.1 Hz), 3.18 (d, 2H, J=17.2 Hz), 3.02 (d, 2H, J=17.2 Hz), 1.81 (s, 1H), 1.13 (t, 3H, J=7.1 Hz) ppm; ¹³C-NMR (CD₃OD) 172.5, 142.7, 133.8, 128.9, 128.8, 128.1, 125.3, 56.9, 56.2, 54.6, 30.4, 17.0 (¹³C signals of the CO ligands were not detected) ppm; LRMS: [M⁺]=716.07 (calc. for C₂₇H₂₇N₇O₅: 715.75); elemental analysis (calculated values in brackets): C 40.22 (40.76), H 3.63 (3.42), N 11.96 (12.32), O 10.50 (10.05), Br 9.07 (10.04; single measurement), Re 24.62 (23.41; calculated).

### Example 14

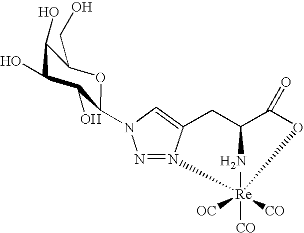

Carbohydrate ligand B1 (22 mg, 0.05 mmol) and [Re(Br)₃(CO)₃][Et₄N]₂ (39 mg, 0.05 mmol) were dissolved in water (3 mL) and the pH was adjusted to pH 7-8 with an aqueous solution of Et₄NOH (10%, 3 drops). The resulting solution was stirred at 50° C. for 3 h. Concentration under reduced pressure followed by HPLC purification of the residue yielded the final product as a white solid (17 mg, 58%): mp>195° C. (decomp.); IR (neat) v cm⁻¹; ¹H-NMR (D₂O) δ 8.31 (s, 1H), 5.82-5.70 (m, 1H), 5.76 (d, 1H, J=9.1 Hz), 5.15 (d, 1H, J=12.0 Hz), 4.35-4.28 (m, 1H), 4.16 (t, 1H, J=9.7 Hz), 4.12 (d, 1H, J=3.2 Hz), 4.05 (t, 1H, J=6.0 Hz), 3.91 (dd, 1H, J=9.7 and 3.3 Hz), 3.87-3.80 (m, 2H), 3.49 (dd, 1H, J=18.2 and 2.4 Hz), 3.45 (dd, 1H, J=28.2 and 4.4 Hz) ppm; ¹³C-NMR (D₂O) δ 197.0, 195.8, 195.4, 184.9, 142.7, 124.9, 88.8, 78.7, 72.7, 69.6, 68.5, 60.8, 51.3, 26.2 ppm; LRLC-MS: [M+H]+=(calc. for C₁₄H₁₇N₄O₁₀: 587.52).

### Example 14

### Labeling of the Ligands L1-13

To the IsoLink™ (Mallinckrodt-Tyco) 1 mL of a 0.9% NaCl solution from a Mo-99/Tc-99m generator is added via the septum. In addition 0.1 mL of stock solution of the corresponding ligand L1-13 or functionalized biomolecules (10⁻⁴ M in saline). The reaction were heated for 30 min at 100° C. and then cooled to room temperature. The solution is neutralized with a solution of PBS (phosphate buffer (pH=7.4, saline 0.9%). Then, the radiochemical composition is studied with HPLC on an RP18 column. The tricarbonyl complex with L1-13 (except L 8) that are produced have considerably longer retention times than [M(OH₂)₃(CO)₃]⁺ (FIGS. 4 and 5)

Example 15 (see also FIGS. 10, 11 and 12): In situ preparation and radiolabeling of triazole-containing metalchelates and biomolecules respectively. In a 10 mL closable vial the following chemicals are put together: 40 microliter of a 10−2 M in H₂O of Cu(II)acetat₂, Na-ascorbat (80 microliter in H₂O, 10⁻² M), 100 microliter of propargyl glycine in H₂O (1012 M). All components can also be added as pure solids into the vial. To this reagents 200 micorliters of 3′-azido thymidine or 1-Azido-1-deoxy-β-D-galactopyranoside (10⁻² M in H₂O). The reaction is heated at 50° C. for 2 h. To the pale yellow solution 100 microliter of [^{99m}Tc(OH₂)₃(CO)₃]⁺ (pH=7.4) was added. The reaction was incubated for 30 min 100° C. and controlled via HPLC. Yield: 84-88%.

1-18. (canceled)

19. A complex with a tricarbonyl-technetium(I) fragment or a tricarbonyl-rhenium(I) fragment and ligands of the general formula (I) 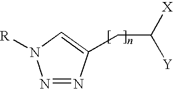
in which n represents numbers 1, 2 and 3;
in which R represents any organic residues;
in which X represents a heteroatom selected from C, O, N, S, P; or a carboxyl, an amino carbonyl, an alkoxycarbonyl, an amino, an aldehyde or an alkoxy group, a heterocyclic residue containing one or more O, N, S, or P atoms;
in which Y represents a heteroatom selected from C, O, N, S, P; or a carboxyl, an amino carbonyl, an alkoxycarbonyl, an amino, an aldehyde or an alkoxy group, a heterocyclic residue containing one or more O, N, S, or P atoms.

20. The complex according to claim 19 wherein said organic residues are biomolecules.

21. The complex according to claim 20 wherein said biomolecules comprise a peptide, a protein, a modified or an unmodified DNA or an RNA oligonucleotide, a nucleotide, a nucleoside, a modified or an unmodified aptamer or a PNA, a vitamin, a carbohydrate, a phospholipid, a receptor for the central nervous system, or combinations thereof.

22. The complex according to claim 19 wherein X represents a primary amine, a secondary amine, or a tertiary amine.

23. The complex according to claim 19 wherein Y represents a primary amine, a secondary amine, or a tertiary amine.

24. A complex with a tricarbonyl-technetium(I) fragment or a tricarbonyl-rhenium(I) fragment and ligands of the general formula (II) 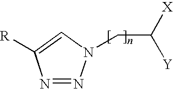
in which n represents numbers 1, 2 and 3;
in which R represents any organic residues;
in which X represents a heteroatom selected from C, O, N, S, P; or a carboxyl, an amino carbonyl, an alkoxycarbonyl, an amino, an aldehyde or an alkoxy group, a heterocyclic residue containing one or more O, N, S, or P atoms; a primary amine, a secondary amine, a tertiary amine;
in which Y represents a heteroatom selected from C, O, N, S, P; or a carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine.

25. The complex according to claim 24 wherein said organic residues are biomolecules.

26. The complex according to claim 25 wherein said biomolecules comprise a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system, or combinations thereof.

27. A complex with a tricarbonyl-technetium(I) fragment or a tricarbonyl-rhenium(I) fragment and ligands of the general formula (III) 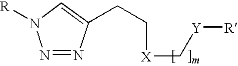
in which m represents numbers 1, 2 and 3;
in which R and/or R′ represents any organic residues;
in which X represents a heteroatom selected from H, C, O, N, S, P; or a halogen or a carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine or biomolecules;
in which Y represents a heteroatom selected from H, C, O, N, S, P; or a halogen, a carboxyl, an amino carbonyl, an alkoxycarbonyl, an amino, an aldehyde or an alkoxy group, a heterocyclic residue containing one or more O, N, S, or P atoms, a primary amine, a secondary amine, or a tertiary amine.

28. The complex according to claim 27 wherein said organic residues are biomolecules.

29. The complex according to claim 28 wherein said biomolecules comprise a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system, or combinations thereof.

30. A complex with a tricarbonyl-technetium(I) fragment or a tricarbonyl-rhenium(I) fragment and ligands of the general formula (IV) 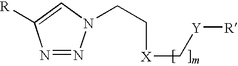
in which m represents numbers 1, 2 and 3;
in which R and/or R′ represents any organic residues;
in which X represents a heteroatom selected from H, C, O, N, S, P; or a halogen or a carboxyl, an amino carbonyl, an alkoxycarbonyl, an amino, an aldehyde or an alkoxy group, a heterocyclic residue containing one or more O, N, S, or P atoms, primary amine, a secondary amine, a tertiary amine or biomolecules;
in which Y represents a heteroatom selected from H, C, O, N, S, P; a halogen, a carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine.

31. The complex according to claim 30 wherein said organic residues are biomolecules.

32. The complex according to claim 31 wherein said biomolecules comprise a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system, or combinations thereof.

33. A complex with a tricarbonyl-technetium(I) fragment or a tricarbonyl-rhenium(I) fragment and ligands of the general formula (V) 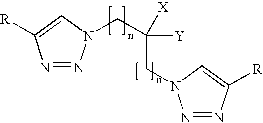
in which n represents numbers 1, 2 and 3;
in which R represents any organic residues;
in which X represents a H or a C atom or heteroatom selected from, O, N, S, P; or a halogen, a carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine or biomolecules;
in which Y represents a H or a C atom or a heteroatom selected from H, C, O, N, S, P; or a halogen, a carboxyl, an amino carbonyl, an alkoxycarbonyl, an amino, an aldehyde or an alkoxy group, a heterocyclic residue containing one or more O, N, S, or P atoms; or a primary amine, a secondary amine, a tertiary amine or biomolecules.

34. The complex according to claim 33 wherein said organic residues are biomolecules.

35. The complex according to claim 34 wherein said biomolecules comprise a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system, or combinations thereof.

36. A complex with a tricarbonyl-technetium(I) fragment or a tricarbonyl-rhenium(I) fragment and ligands of the general formula (VI) 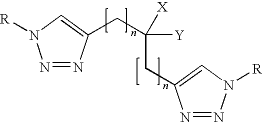
in which n represents numbers 1, 2 and 3;
in which R represents any organic residues;
in which X represents a H or a C atom or a heteroatom selected from, O, N, S, P; or a halogen or a carboxyl, an amino carbonyl, an alkoxycarbonyl, an amino, an aldehyde or an alkoxy group, a heterocyclic residue containing one or more O, N, S, or P atoms, primary amine, a secondary amine, a tertiary amine or biomolecules;
in which Y represents a H or a C atom or a heteroatom selected from O, N, S, P; or a halogen, a carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine or biomolecules.

37. The complex according to claim 36 wherein said organic residues are biomolecules.

38. The complex according to claim 37 wherein said biomolecules comprise a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system, or combinations thereof.

39. A complex with a tricarbonyl-technetium(I) fragment or a tricarbonyl-rhenium(I) fragment and ligands of the general formula (VII) 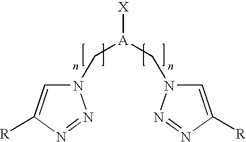
in which n represents numbers 1, 2;
in which R represents any organic residues;
in which A represents a heteroatom selected from H, C, O, N, S, P, As;
in which X represents a carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine, or biomolecules.

40. The complex according to claim 39 wherein said organic residues are biomolecules.

41. The complex according to claim 40 wherein said biomolecules comprise a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system, or combinations thereof.

42. A complex with a tricarbonyl-technetium(I) fragment or a tricarbonyl-rhenium(I) fragment and ligands of the general formula (VIII) 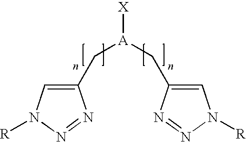
in which n represents numbers 1, 2;
in which R represents any organic residues;
in which A represents a heteroatom selected from H, C, O, N, S, P, As;
in which X represents a carboxyl, an amino carbonyl, an alkoxycarbonyl, an amino, an aldehyde or an alkoxy group, a heterocyclic residue containing one or more O, N, S, or P atoms, primary amine, a secondary amine, a tertiary amine or biomolecules.

43. The complex according to claim 42 wherein said organic residues are biomolecules.

44. The complex according to claim 43 wherein said biomolecules comprise a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system, or combinations thereof.

45. A process for the in situ production of complexes with the tricarbonyl-technetium(I) fragment or the tricarbonyl-rhenium(I) fragment with the compound of general formula (I) according to claim 19, in that a compound of the general formula 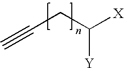
in which n represents numbers 1, 2 and 3;
in which X represents a heteroatom selected from C, O, N, S, P; or a carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine or biomolecules;
in which Y represents a heteroatom selected from C, O, N, S, P; or a carboxyl, an amino carbonyl, an alkoxycarbonyl, an amino, an aldehyde or an alkoxy group, a heterocyclic residue containing one or more O, N, S, or P atoms, primary amine, a secondary amine, a tertiary amine or biomolecules;
is reacted with a compound of general formula
R—N₃
in which R represents any organic residues in particular biomolecules e.g. a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system;
according to the reaction:

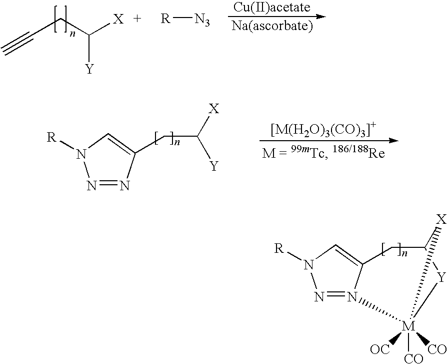

46. The process according to claim 45 in which said biomolecules are a peptide, a protein, a modified or an unmodified DNA or a RNA oligonucleotide, a nucleotide, a nucleoside, a modified or an unmodified aptamer or a PNA, a vitamin, a carbohydrate, a phospholipid, or a receptor for the central nervous system.

47. A process for the in situ production of complexes with the tricarbonyl-technetium(I) fragment or the tricarbonyl-rhenium(I) fragment with the compound of general formula (II) according to claim 25, in that compound of the general formula 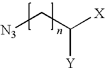
in which n represents numbers 1, 2 and 3;
in which X represents a heteroatom selected from C, O, N, S, P; or a carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine, or biomolecules;
in which Y represents a heteroatom selected from C, O, N, S, P or a carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine or biomolecules;
is reacted with a compound of general formula 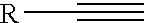
in which R represents any organic residues;
according to the following reaction diagram:

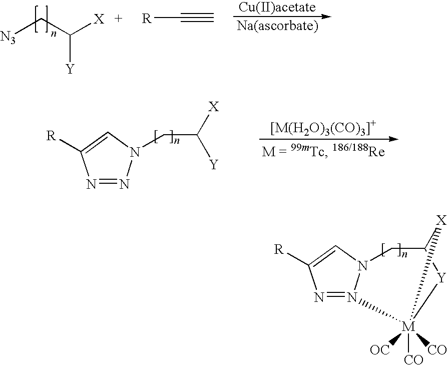

48. The process according to claim 47 wherein said biomolecules are a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system.

49. The process according to claim 47 wherein said organic residues are biomolecules.

50. The process according to claim 49 wherein said biomolecules are a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system, or combinations thereof.

51. A process for the in situ production of complexes with the tricarbonyl-technetium(I) fragment or the tricarbonyl-rhenium(I) fragment with the compound of general formula (III) according to claim 27, in that a compound of the general formula 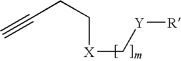
in which m represents numbers 2 and 3;
in which R′ represents H or any organic residues or biomolecules;
in which X represents a heteroatom selected from C, O, N, S, P; or a carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine, or biomolecules;
in which Y represents a heteroatom selected from C, O, N, S, P; or a carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine or biomolecules;
is reacted with a compound of general formula
R—N₃
in which R represents any organic residues or biomolecules e.g. a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system;
according to the following reaction diagram:

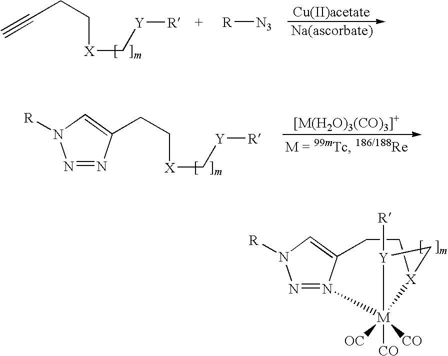

52. The process according to claim 51 wherein said biomolecules are a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system.

53. The process according to claim 51 wherein said organic residues are biomolecules.

54. The process according to claim 53 wherein said biomolecules are a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system, or combinations thereof.

55. A process for the in situ production of complexes with the tricarbonyl-technetium(I) fragment or the tricarbonyl-rhenium(I) fragment with the compound of general formula (IV) according to claim 30, in that compound of the general formula 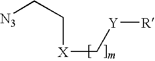
in which m represents numbers 2 and 3;
in which R′ represents H or any organic residues;
in which X represents a heteroatom selected from C, O, N, S, P; or a carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine or;
in which Y represents a heteroatom selected from C, O, N, S, P; or a carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine or biomolecules;
is reacted with a compound of general formula 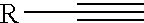
in which R represents any organic residues or biomolecules;
according to the following reaction diagram:

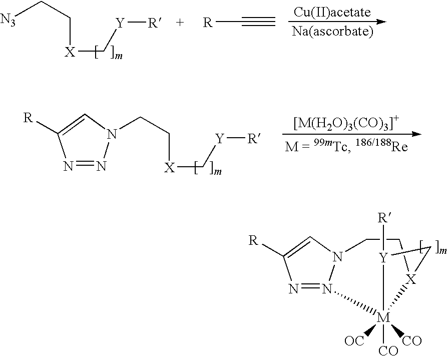

56. The process according to claim 55 wherein said biomolecules are a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system.

57. The process according to claim 55 wherein said organic residues are biomolecules.

58. The process according to claim 57 wherein said biomolecules are a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system, or combinations thereof.

59. A process for the in situ production of complexes with the tricarbonyl-technetium(I) fragment or the tricarbonyl-rhenium(I) fragment with the compound of general formula (V) according to claim 33, in that compound of the general formula 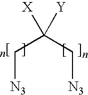
in which n represents numbers 1, 2;
in which X represents a H or a C atom or a heteroatom selected from, O, N, S, P; or a halogen, a carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine or biomolecules;
in which Y represents a H or a C atom or a heteroatom selected from O, N, S, P; or a halogen, a carboxyl, an amino carbonyl, an alkoxycarbonyl, an amino, an aldehyde or an alkoxy group, a heterocyclic residue containing one or more O, N, S, or P atoms, primary amine, a secondary amine, a tertiary amine or biomolecules;
is reacted with a compound of general formula 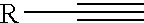
in which R represents any organic residues;
according to the following reaction diagram:

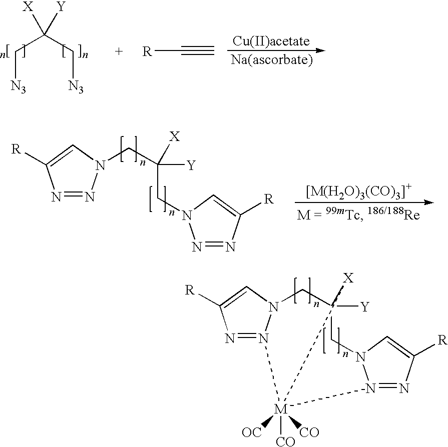

60. The process according to claim 59 wherein said biomolecules are a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system.

61. The process according to claim 59 wherein said organic residues are biomolecules.

62. The process according to claim 61 wherein said biomolecules are a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system, or combinations thereof.

63. A process for the in situ production of complexes with the tricarbonyl-technetium(I) fragment or the tricarbonyl-rhenium(I) fragment according to claim 36 in that compound of the general formula 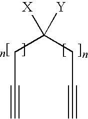
in which n represents numbers 1, 2;
in which X represents a H or a C atom or a heteroatom selected from, O, N, S, P; or a halogen or a carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine or biomolecules;
in which Y represents a H or a C atom or a heteroatom selected from O, N, S, P; or a halogen or a or a carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine or biomolecules;
is reacted with a compound of general formula
R—N₃
in which R represent any organic residues or biomolecules;
according to the following reaction diagram:

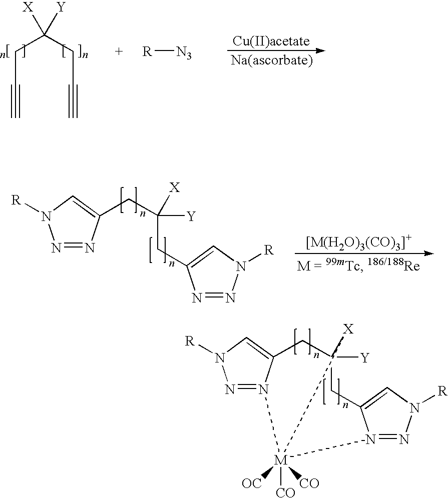

64. The process according to claim 63 wherein said biomolecules are a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system.

65. The process according to claim 63 wherein said organic residues are biomolecules.

66. The process according to claim 65 wherein said biomolecules are a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system, or combinations thereof.

67. A process for the in situ production of complexes with the tricarbonyl-technetium(I) fragment or the tricarbonyl-rhenium(I) fragment with the compound of general formula (VII) according to claim 39, in that compound of the general formula 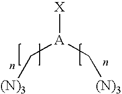
in which n represents numbers 1, 2;
in which A represents a heteroatom selected from H, C, O, N, S, P, As;
in which X represents carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine or a biomolecules;
is reacted with a compound of general formula 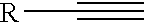
in which R represents any organic residues or biomolecules;
according to the following reaction diagram:

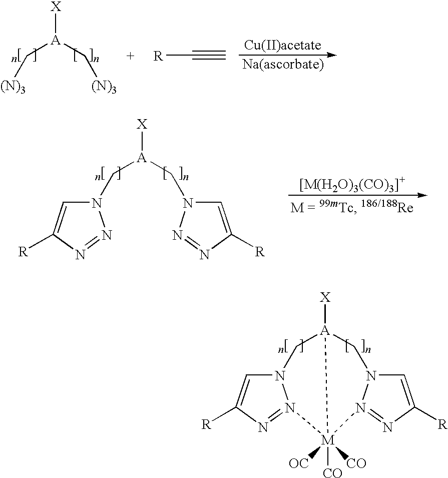

68. The process according to claim 67 wherein said biomolecules are a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system.

69. The process according to claim 67 wherein said organic residues are biomolecules.

70. The process according to claim 69 wherein said biomolecules are a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system, or combinations thereof.

71. A process for the in situ production of complexes with the tricarbonyl-technetium(I) fragment or the tricarbonyl-rhenium(I) fragment with the compound of general formula (VIII) according to claim 42, in that compound of the general formula 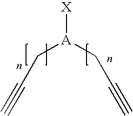
in which n represents numbers 1, 2;
in which A represents a heteroatom selected from H, C, O, N, S, P, As;
in which X is a carboxyl, an amino carbonyl, an alkoxycarbonyl, an amino, an aldehyde or an alkoxy group, a heterocyclic residue containing one or more O, N, S, or P atoms, a primary amine, a secondary amine, a tertiary amine or a biomolecules;
is reacted with a compound of general formula
R—N₃
in which R represents any organic residues or biomolecules
according to the following reaction diagram:

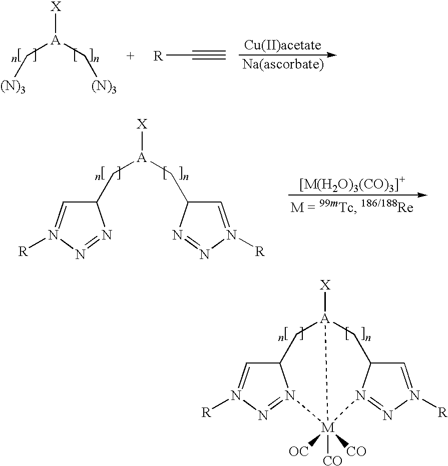

72. The process according to claim 71 wherein said biomolecules are a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system.

73. The process according to claim 71 wherein said organic residues are biomolecules.

74. The process according to claim 73 wherein said biomolecules are a peptide, a protein, a modified or an unmodified DNA or a RNA oligonucleotide, a nucleotide, a nucleoside, a modified or an unmodified aptamer or a PNA, a vitamin, a carbohydrate, a phospholipid, a receptor for the central nervous system, or combinations thereof.

75. A kit for the preparation of tricarbonyl-technetium(I) fragments or a tricarbonyl-rhenium(I) fragments, ligands, complexes and derivatives thereof based on an IsoLink technology with ^{99m}Tc.(i) a metal selected from the group consisting of, ^{186/188}Re and their permetallate: (ii) a reducing agent soluble in water but not substantially decomposed by water, (iii) a base, (iv) optionally, a stabilizing agent and/or chelator, (v) optionally, one or more inert pharmaceutically acceptable carriers and/or formulating agents and/or adjuvants, at least one of said ingredients being stored in a container having an atmosphere containing a sufficient amount of carbon monoxide to form a complex of a general formula ML(CO)₃

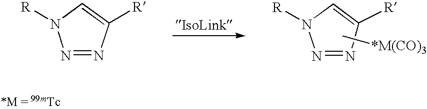

76. A kit for the preparation of tricarbonyl-technetium(I) fragments or a tricarbonyl-rhenium(I) fragments, ligands, complexes, and derivatives thereof comprising: (i) a metal selected from the group consisting of, ^{99m}Tc or ^{186/188}Re and their permetallate (ii) a reducing agent soluble in water but not substantially decomposed by water, (iii) a base, (iv) if desired, a stabilizing agent and/or chelator, (v) if desired one or more inert pharmaceutically acceptable carriers and/or formulating agents and/or adjuvants, at least one of said ingredients being stored in a container having an atmosphere containing a sufficient amount of potassium boranocarbonate to form a complex of a general formula ML(CO)₃ [chemical structure]
